(19) United States
(12) Patent Application Publication
(10) Pub. No.: US 2023/0201369 A1
(43) Pub. Date: Jun. 29, 2023

(54) NALOXONE NANOPARTICLE COMPOSITIONS AND METHODS THEREOF

(71) Applicant: THE TEXAS A&M UNIVERSITY SYSTEM, College Station, TX (US)

(72) Inventors: Shoshana EITAN, College Station, TX (US); Naga Venkata Ravi Kumar MAJETI, College Station, TX (US)

(21) Appl. No.: 17/996,562

(22) PCT Filed: Apr. 29, 2021
(86) PCT No.: PCT/US2021/029867

§ 371 Date: Oct. 19, 2022

Related U.S. Application Data
(60) Provisional application No. 63/017,228, filed on Apr. 29, 2020.

(51) Int. Cl. A61K 47/69; A61K 31/485; A61P 25/36

(52) U.S. Cl. CPC A61K 47/6937; A61K 31/485; A61P 25/36; A61K 9/0053

(57) ABSTRACT

The present disclosure provides nanoparticle compositions comprising i) a polymeric nanoparticle, ii) one or more ligands conjugated to the polymeric nanoparticle, and iii) naloxone. The disclosure also provides methods and pharmaceutical compositions comprising the nanoparticle compositions for use in treating patients with various disease states.

### CROSS-REFERENCE TO RELATED APPLICATIONS

This application claims the benefit under 35 USC § 119(e) of U.S. Provisional Application Ser. No. 63/017,228, filed on Apr. 29, 2020, the entire disclosure of which is incorporated herein by reference.

### BACKGROUND AND SUMMARY OF THE INVENTION

Opioids are a class of drugs which are routinely prescribed to alleviate moderate-to-severe pain. Unfortunately, opioids are highly reinforcing, and liable for dependence, abuse, and addiction. Opioid overdose, causing severe opioid-induced respiratory depression (OIRD), accounts for the death of more than 130 Americans every day.

Naloxone, a nonspecific antagonist of the mu-opioid receptors, is used for the reversal of OIRD. Naloxone is available in different formulations such as prepackaged nasal spray and as injectable administration, including intravenous, intramuscular, and subcutaneous. However, an oral formulation of naloxone is not currently available due to the oral bioavailability of naloxone being very low (e.g., estimated to be 0.9-2%).

Furthermore, one of the major challenges of using naloxone is its short elimination half-life, estimated to be about 30-40 minutes. The half-life of naloxone is significantly shorter compared to many opioid analgesics (i.e. agonists). Thus, renarcotization, and rapid return to full respiratory depression, might occur within 30-45 minutes after a single dose of naloxone, particularly in individuals who have taken large doses or long-acting opioid formulations.

Increasing naloxone's duration of action can be achieved by administering higher doses. However, naloxone may cause serious and possibly life-threatening side effects in some individuals. Specifically, naloxone in high doses or, if infused rapidly, can cause pulmonary edema, cardiac arrhythmias, hypertension, and cardiac arrest in patients who are hypovolemic, hypotensive, and/or suffering from severe pain or stress. These complications are due to a naloxone-induced massive release of catecholamines. However, to counteract newer synthetic opioids that have high receptor affinity (e.g., fentanyl and long-acting opioid formulations), a greater naloxone concentration and/or a continuous infusion is required as compared with what is required to counteract an opioid with lower receptor affinity.

Therefore, the clinical approach to reverse severe OIRD and prevent the recurrence of renarcotization, specifically caused by fentanyl and other ultra-potent opioids, requires practitioners to use higher naloxone doses, very careful titration regimens, or continuous infusion until chances for renarcotization have diminished. Effective but safe dosing of naloxone is challenging, and even more so in an out-of-hospital setting and by an untrained bystander.

Moreover, although prior nanoparticle formulations of naloxone have been tested in preclinical models, they have been limited to injectable formulations and not appropriate for oral administration. Therefore, there exists a need for new compositions and methods that are efficacious in delivering naloxone to patients for therapeutic uses such as OIRD and other indications for which naloxone is utilized.

Accordingly, the present disclosure provides nanoparticle compositions comprising naloxone that can be formulated for improved delivery to patients, including oral administration. Furthermore, the present disclosure provides several methods of administering the nanoparticle compositions to patients in various disease states for which naloxone can be therapeutically beneficial.

The compositions and methods of the present disclosure provide several advantages compared to the current state of the art. First, the nanoparticle compositions are capable of being formulated for oral administration for improved delivery of naloxone to patients. Furthermore, the nanoparticle compositions can be utilized in methods of treating patients with OIRD. Finally, the nanoparticle compositions can be utilized in methods of treating other disease states for which naloxone may be therapeutically beneficial. The methods include treatment of patients with opioid use disorder, alcoholism, opioid overdose, post-operative opioid depression, hypertension, pruritus, and urinary retention, among others.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1C show the characterization of the nanoparticle compositions. FIG. 1A shows schematic representation of periodic functional polyester synthesis. FIG. 1B shows dynamic light scattering (DLS) intensity distribution plots of naloxone-laden particles (NP-Naloxone), inset is a model particle with purple dots representing naloxone. FIG. 3C shows scanning electron photomicrographs of naloxone-laden particles (NP Naloxone).

FIGS. 2A-2C show a schematic representation of the experimental design. FIG. 2A shows duration of action; FIG. 2B shows withdrawal precipitation; and FIG. 2C shows onset of action.

FIG. 3 shows duration of action antagonizing morphine-induced locomotion, total distance traveled results. Baseline (BL) locomotor activity of each mouse was recorded for 30 minutes. Then, mice were administered orally (via gavage) with NP control, NP (Naloxone) 1 mg/kg, or NP (Naloxone) 5 mg/kg. Mice were injected with 10 mg/kg morphine (s.c.) immediately (T0), 10 hours (T10), 24 hours (T24), 34 hours (T34), 48 hours (T48), 72 hours (T72), and 96 hours (T96) following the NP administration. Each time mice were recorded for 3 hours. (*) indicates a significant difference from NP control group (p<0.05). Results for the total distance traveled during each period are presented as mean±SEM.

FIGS. 4A-4G show duration of action—antagonizing morphine-induced locomotion, temporal representation results. Baseline (BL; see FIG. 4A) locomotor activity of each mouse was recorded for 30 minutes. Then, mice were administered orally (via gavage) with NP control, NP (Naloxone) 1 mg/kg, or NP (Naloxone) 5 mg/kg. Mice were injected with 10 mg/kg morphine (s.c.) immediately (T0; see FIG. 4A), 10 hours (T10; see FIG. 4B), 24 hours (T24; see FIG. 4C), 34 hours (T34; see FIG. 4D), 48 hours (T48; see FIG. 4E), 72 hours (T72; see FIG. 4F), and 96 hours (T96; see FIG. 4G) following the NP administration. Each time they were recorded for 3 hours. Results for the 5-minute intervals during each of the periods are presented as mean±SEM FIGS. 5A-5E show duration of action—antagonizing morphine-induced antinociception results. Mice were administered orally (via gavage, 10 ml/kg) with NP control or 5 mg/kg NP Naloxone. The ability of NP Naloxone to antagonize the antinociceptive effects of 10 mg/kg morphine was examined immediately after (T0; see FIG. 5A), 24 hours (T24; see FIG. 5B), 48 hours (T48; see FIG. 5C), 72 hours (T72; see FIG. 5D), and 120 hours (T120; see FIG. 5E) following the NP administration. (*) indicates a significant difference from NP control group (p<0.05). (§) indicates a significant difference from NP control group (p<0.001). Results for the total distance traveled during each period are presented as mean±SEM.

FIGS. 6A-6B show the results of withdrawal precipitation. Mice were administered with escalating doses of morphine for 6 days. On day 7, mice were administrated with 20 mg/kg morphine. Two hours later, mice were administered with naloxone (1 or 5 mg/kg, i.p.) or orally (via gavage) with NP control, NP (Naloxone) 1 mg/kg, or NP (Naloxone) 5 mg/kg. The numbers of jumps in the following 30 min were scored. (*) indicates a significant difference (p<0.05). Results are presented as mean±SEM (FIG. 6A) and as individuals scores (FIG. 6B).

FIG. 7 shows the results of onset of action. Mice were recorded for baseline activity, administered with morphine and recorded for another 60 minutes, then administered with NP control (dotted black line) or NP (Naloxone) 1 mg/kg (broken red line) and recorded for 10 minutes. Perpendicular black broken line notes the time of NP administration. The differences between the 1-minute intervals of minutes 26-30 of baseline (BL), the 56-60 minutes post-morphine administration, and the 10 minutes post-NP administration (i.e. the 61-70 minutes post-morphine administration) were computed. (ϕ) indicates a significant difference from minute 30 of BL (p<0.05), (ε) indicates a significant difference from minute 60 post-morphine (p<0.05), (*) indicates a significant difference from NP controls (p<0.05). Results are presented as mean+SEM.

### DETAILED DESCRIPTION

Various embodiments of the invention are described herein as follows. In an illustrative aspect, a nanoparticle composition is provided. The nanoparticle composition comprises i) a polymeric nanoparticle, ii) one or more ligands conjugated to the polymeric nanoparticle, and iii) naloxone.

Naloxone, also known as N-Allylnoroxymorphone; 17-Allyl-4,5α-epoxy-3,14-dihydroxymorphinan-6-one by its IUPAC name 3,8-Dihydroxybenzo[c]chromen-6-one, is a compound that is a morphinan derivative. The chemical structure of naloxone is:

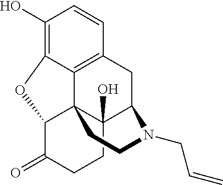

As used herein, the term “naloxone” refers to naloxone base, pharmaceutically acceptable salts of naloxone, other salts of naloxone, and metabolites of naloxone. The term “pharmaceutically acceptable salt” refers to an addition salt that exists in conjunction with the acidic or basic portion of naloxone. Such salts include the pharmaceutically acceptable salts listed in HANDBOOK OF PHARMACEUTICAL SALTS: PROPERTIES, SELECTION AND USE, P. H. Stahl and C. G. Wermuth (Eds.), Wiley-VCH, New York, 2002 which are known to the skilled artisan. Pharmaceutically acceptable salts of an acid addition nature are formed when naloxone and any of its intermediates containing a basic functionality are reacted with a pharmaceutically acceptable acid. Pharmaceutically acceptable acids commonly employed to form such acid addition salts include inorganic and organic acids. Pharmaceutically acceptable salts of a base addition nature are formed when naloxone and any of its intermediates containing an acidic functionality are reacted with a pharmaceutically acceptable base. Pharmaceutically acceptable bases commonly employed to form base addition salts include organic and inorganic bases.

In addition to pharmaceutically acceptable salts, other salts are included in the present invention. They may serve as intermediates in the purification of compounds or in the preparation of other pharmaceutically-acceptable salts, or are useful for identification, characterization or purification.

In an embodiment, the polymeric nanoparticle comprises a polymer/copolymer selected from the group consisting of polylactide, poly(lactide-co-glycolide), polycaprolactone, and any combination thereof.

In an embodiment, the naloxone and the polymer/copolymer are present at a ratio of about 1:4 (naloxone:polymer/copolymer). In an embodiment, the naloxone and the polymer/copolymer are present at a ratio of about 1:5 (naloxone:polymer/copolymer). In an embodiment, the naloxone and the polymer/copolymer are present at a ratio of about 1:10 (naloxone:polymer/copolymer). In an embodiment, the naloxone and the polymer/copolymer are present at a ratio of about 1:12 (naloxone:polymer/copolymer). In an embodiment, the naloxone and the polymer/copolymer are present at a ratio of about 1:15 (naloxone:polymer/copolymer). In an embodiment, the naloxone and the polymer/copolymer are present at a ratio of about 1:20 (naloxone:polymer/copolymer). In an embodiment, the naloxone and the polymer/copolymer are present at a ratio of about 1:25 (naloxone:polymer/copolymer). In an embodiment, the naloxone and the polymer/copolymer are present at a ratio of about 0.1 mg:1 mg (naloxone:polymer/copolymer). In an embodiment, the naloxone and the polymer/copolymer are present at a ratio of about 1 mg:10 mg (naloxone:polymer/copolymer). In an embodiment, the naloxone and the polymer/copolymer are present at a ratio of about 0.5 mg:5 mg (naloxone:polymer/copolymer).

In an embodiment, the ligand is gambogic acid. It is well known in the art that gambogic acid (GA) is a noncompetitive ligand of the transferrin receptor (TfR1). Without being bound by any theory, it is believed that conjugation of GA to polymeric nanoparticles can facilitate transfer of the polymeric nanoparticles through the gastrointestinal barrier via TFRC (Transferrin Receptor 1) interaction. Furthermore, GA binding does not appear to interfere with normal transferrin-mediated iron metabolism to advantageously provide intestinal transport of the polymeric nanoparticles. Moreover, without being bound by any theory, it is also believed that conjugation of GA to polymeric nanoparticles can facilitate transfer of the polymeric nanoparticles across the blood-brain barrier (BBB).

In an embodiment, the naloxone is encapsulated by the polymeric nanoparticle.

The term “nanoparticle” refers to a particle having a size measured on the nanometer scale. As used herein, the “nanoparticle” refers to a particle having a structure with a size of less than about 1,000 nanometers. As used herein, the term “nanoparticle composition” refers to any substance that contains at least one nanoparticle. In some embodiments, a nanoparticle composition is a uniform collection of nanoparticles.

Methods for formulation of nanoparticle compositions using polymer/copolymer and/or GA can be found, for example, in U.S. Patent Application Publication No. 2018/0110865 and U.S. Patent Application Publication No. 2018/0214386, both herein incorporated by reference in their entireties.

In an embodiment, the nanoparticle composition has an average diameter from about 0.5 nm to about 1000 nm. Particle sizes are determined by methods well known in the art, such as by dynamic light scattering, SEM. In an embodiment, the nanoparticle composition has an average diameter from about 1 nm to about 500 nm. In an embodiment, the nanoparticle composition has an average diameter from about 10 nm to about 400 nm. In an embodiment, the nanoparticle composition has an average diameter from about 100 nm to about 400 nm. In an embodiment, the nanoparticle composition has an average diameter from about 100 nm to about 300 nm. In an embodiment, the nanoparticle composition has an average diameter from about 100 nm to about 200 nm. In an embodiment, the nanoparticle composition has an average diameter from about 100 nm to about 150 nm. In an embodiment, the nanoparticle composition has an average diameter from about 200 nm to about 400 nm. In an embodiment, the nanoparticle composition has an average diameter from about 300 nm to about 400 nm. In an embodiment, the nanoparticle composition has an average diameter from about 150 nm to about 300 nm. In an embodiment, the nanoparticle composition has an average diameter from about 150 nm to about 200 nm. In an embodiment, the nanoparticle composition has an average diameter from about 200 nm to about 300 nm. In an embodiment, the nanoparticle composition has an average diameter from about 250 nm to about 300 nm.

In an embodiment, the nanoparticle composition is lyophilized.

In an illustrative aspect, a pharmaceutical composition is provided. The pharmaceutical composition comprises a nanoparticle composition, wherein the nanoparticle composition comprises i) a polymeric nanoparticle, ii) one or more ligands conjugated to the polymeric nanoparticle, and iii) naloxone.

The previously described embodiments of the nanoparticle composition are also applicable to the pharmaceutical composition.

In an embodiment, the pharmaceutical composition is an oral formulation. In an embodiment, the oral formulation is selected from the group consisting of a tablet, a capsule, a suspension, an emulsion, a syrup, a colloidal dispersion, a dispersion, and an effervescent composition. In an embodiment, the oral formulation is a suspension. In an embodiment, the oral formulation is a reconstitutable suspension.

In an embodiment, the pharmaceutical composition is a parenteral formulation. In an embodiment, the parenteral formulation is selected from the group consisting of intravenous, intraarterial, intraperitoneal, intrathecal, intradermal, epidural, intracerebroventricular, intraurethral, intrasternal, intracranial, intratumoral, intramuscular and subcutaneous.

In an embodiment, the pharmaceutical composition comprises one or more pharmaceutically acceptable carriers. Such pharmaceutically acceptable carriers include those listed in HANDBOOK OF PHARMACEUTICAL EXCIPIENTS, P. J. Sheskey et al. (Eds.), Pharmaceutical Press, 2017 which are known to the skilled artisan.

In an embodiment, the pharmaceutical composition further comprises a second therapeutic agent. In some aspects, the pharmaceutical composition is adapted for administration with a second therapeutic agent. The second therapeutic agent can comprise a compound disclosed herein or a compound, pharmaceutical, or other chemical entity that is shown to be therapeutically effective in treating or affecting one or more disease state of the present disclosure.

In an embodiment, the pharmaceutical composition is formulated as a single dose. In an embodiment, the pharmaceutical composition is a single unit dose. As used herein, the term “unit dose” is a discrete amount of the composition comprising a predetermined amount of the compound. The amount of the compound is generally equal to a dosage which would be administered to an animal or a convenient fraction of such a dosage such as, for example, one-half or one-third of such a dosage.

In an illustrative aspect, a method of treating opioid induced respiratory depression in a patient in need thereof is provided. The method comprises the step of administering a therapeutically effective amount of a nanoparticle composition to the patient, wherein the nanoparticle composition comprises i) a polymeric nanoparticle, ii) one or more ligands conjugated to the polymeric nanoparticle, and iii) naloxone.

The previously described embodiments of the nanoparticle composition and the pharmaceutical composition are also applicable to the method of treating opioid induced respiratory depression. In an embodiment, the patient is an animal. In an embodiment, the animal is a mammal. In an embodiment, the animal is a human.

In an embodiment, the nanoparticle composition is administered to the patient at a dose of about 0.001 to about 1000 mg of the nanoparticle composition per kg of body weight. In an embodiment, the nanoparticle composition is administered to the patient at a dose of about 0.001 to about 100 mg of the nanoparticle composition per kg of body weight. In an embodiment, the nanoparticle composition is administered to the patient at a dose of about 0.001 to about 10 mg of the nanoparticle composition per kg of body weight. In an embodiment, the nanoparticle composition is administered to the patient at a dose of about 1 to about 5 mg of the nanoparticle composition per kg of body weight. In an embodiment, the nanoparticle composition is administered to the patient at a dose of about 1 mg of the nanoparticle composition per kg of body weight. In an embodiment, the nanoparticle composition is administered to the patient at a dose of about 2 mg of the nanoparticle composition per kg of body weight. In an embodiment, the nanoparticle composition is administered to the patient at a dose of about 3 mg of the nanoparticle composition per kg of body weight. In an embodiment, the nanoparticle composition is administered to the patient at a dose of about 4 mg of the nanoparticle composition per kg of body weight. In an embodiment, the nanoparticle composition is administered to the patient at a dose of about 5 mg of the nanoparticle composition per kg of body weight.

In an embodiment, the administration is an oral administration. In an embodiment, the oral administration is selected from the group consisting of a tablet, a capsule, a suspension, an emulsion, a syrup, a colloidal dispersion, a dispersion, and an effervescent composition. Oral administration can preferably be performed utilizing a suspension, for example via a reconstituted suspension.

In an embodiment, the administration is a parenteral administration. In an embodiment, the parenteral administration is selected from the group consisting of intravenous, intraarterial, intraperitoneal, intrathecal, intradermal, epidural, intracerebroventricular, intraurethral, intrasternal, intracranial, intratumoral, intramuscular and subcutaneous.

In an embodiment, the nanoparticle composition is administered as a single dose. In an embodiment, the nanoparticle composition is administered as a single unit dose. In an embodiment, the method further comprises administration of a second therapeutic agent to the patient.

In an embodiment, the nanoparticle composition is administered to the patient once daily. In an embodiment, the nanoparticle composition is administered to the patient twice daily. In an embodiment, the nanoparticle composition is administered to the patient four times per week. In an embodiment, the nanoparticle composition is administered to the patient three times per week. In an embodiment, the nanoparticle composition is administered to the patient two times per week. In an embodiment, the nanoparticle composition is administered to the patient one time per week. In an embodiment, the nanoparticle composition is administered to the patient every 10 days. In an embodiment, the nanoparticle composition is administered to the patient every 14 days. In an embodiment, the nanoparticle composition is administered to the patient every 15 days. In an embodiment, the nanoparticle composition is administered to the patient every 21 days. In an embodiment, the nanoparticle composition is administered to the patient every 28 days. In an embodiment, the nanoparticle composition is administered to the patient one time per month.

In an embodiment, the nanoparticle composition provides an onset of action to the patient in about 5 minutes. In an embodiment, the nanoparticle composition provides an onset of action to the patient in about 10 minutes. In an embodiment, the nanoparticle composition provides an onset of action to the patient in about 15 minutes. In an embodiment, the nanoparticle composition provides an onset of action to the patient in about 20 minutes. In an embodiment, the nanoparticle composition provides an onset of action to the patient in about 25 minutes. In an embodiment, wherein the nanoparticle composition provides an onset of action to the patient in about 30 minutes. In an embodiment, the nanoparticle composition provides an onset of action to the patient in about 35 minutes. In an embodiment, the nanoparticle composition provides an onset of action to the patient in about 40 minutes. In an embodiment, the nanoparticle composition provides an onset of action to the patient in about 45 minutes. In an embodiment, the nanoparticle composition provides an onset of action to the patient in about 60 minutes.

In an illustrative aspect, a method of treating opioid use disorder in a patient in need thereof is provided. The method comprises the step of administering a therapeutically effective amount of a nanoparticle composition to the patient, wherein the nanoparticle composition comprises i) a polymeric nanoparticle, ii) one or more ligands conjugated to the polymeric nanoparticle, and iii) naloxone. The previously described embodiments of the nanoparticle composition, the pharmaceutical composition, and the described methods are also applicable to the method of treating opioid use disorder.

In an illustrative aspect, a method of treating alcoholism in a patient in need thereof is provided. The method comprises the step of administering a therapeutically effective amount of a nanoparticle composition to the patient, wherein the nanoparticle composition comprises i) a polymeric nanoparticle, ii) one or more ligands conjugated to the polymeric nanoparticle, and iii) naloxone. The previously described embodiments of the nanoparticle composition, the pharmaceutical composition, and the described methods are also applicable to the method of treating alcoholism.

In an illustrative aspect, a method of treating opioid overdose in a patient in need thereof is provided. The method comprises the step of administering a therapeutically effective amount of a nanoparticle composition to the patient, wherein the nanoparticle composition comprises i) a polymeric nanoparticle, ii) one or more ligands conjugated to the polymeric nanoparticle, and iii) naloxone. The previously described embodiments of the nanoparticle composition, the pharmaceutical composition, and the described methods are also applicable to the method of treating opioid overdose.

In an illustrative aspect, a method of treating post-operative opioid depression in a patient in need thereof is provided. The method comprises the step of administering a therapeutically effective amount of a nanoparticle composition to the patient, wherein the nanoparticle composition comprises i) a polymeric nanoparticle, ii) one or more ligands conjugated to the polymeric nanoparticle, and iii) naloxone. The previously described embodiments of the nanoparticle composition, the pharmaceutical composition, and the described methods are also applicable to the method of treating post-operative opioid depression.

In an illustrative aspect, a method of treating hypertension in a patient in need thereof is provided. The method comprises the step of administering a therapeutically effective amount of a nanoparticle composition to the patient, wherein the nanoparticle composition comprises i) a polymeric nanoparticle, ii) one or more ligands conjugated to the polymeric nanoparticle, and iii) naloxone. The previously described embodiments of the nanoparticle composition, the pharmaceutical composition, and the described methods are also applicable to the method of treating hypertension. In an embodiment, the hypertension is associated with management of septic shock in the patient.

In an illustrative aspect, a method of treating pruritus in a patient in need thereof is provided. The method comprises the step of administering a therapeutically effective amount of a nanoparticle composition to the patient, wherein the nanoparticle composition comprises i) a polymeric nanoparticle, ii) one or more ligands conjugated to the polymeric nanoparticle, and iii) naloxone. The previously described embodiments of the nanoparticle composition, the pharmaceutical composition, and the described methods are also applicable to the method of treating pruritus. In an embodiment, the pruritus is opioid-induced pruritus.

In an illustrative aspect, a method of preventing urinary retention in a patient in need thereof is provided. The method comprises the step of administering a therapeutically effective amount of a nanoparticle composition to the patient, wherein the nanoparticle composition comprises i) a polymeric nanoparticle, ii) one or more ligands conjugated to the polymeric nanoparticle, and iii) naloxone. The previously described embodiments of the nanoparticle composition, the pharmaceutical composition, and the described methods are also applicable to the method of preventing urinary retention. In an embodiment, the patient is a post-operative patient. In an embodiment, the post-operative patient utilizes a patient controlled analgesia (PCA) device.

The following numbered embodiments are contemplated and are non-limiting:
1. A nanoparticle composition comprising i) a polymeric nanoparticle, ii) one or more ligands conjugated to the polymeric nanoparticle, and iii) naloxone.
2. The nanoparticle composition of clause 1, any other suitable clause, or any combination of suitable clauses, wherein the polymeric nanoparticle comprises a polymer/copolymer selected from the group consisting of polylactide, poly(lactide-co-glycolide), polycaprolactone, and any combination thereof.
3. The nanoparticle composition of clause 2, any other suitable clause, or any combination of suitable clauses, wherein the naloxone and the polymer/copolymer are present at a ratio of about 1:4 (naloxone:polymer/copolymer).
4. The nanoparticle composition of clause 2, any other suitable clause, or any combination of suitable clauses, wherein the naloxone and the polymer/copolymer are present at a ratio of about 1:5 (naloxone:polymer/copolymer).
5. The nanoparticle composition of clause 2, any other suitable clause, or any combination of suitable clauses, wherein the naloxone and the polymer/copolymer are present at a ratio of about 1:10 (naloxone:polymer/copolymer).
6. The nanoparticle composition of clause 2, any other suitable clause, or any combination of suitable clauses, wherein the naloxone and the polymer/copolymer are present at a ratio of about 1:12 (naloxone:polymer/copolymer).
7. The nanoparticle composition of clause 2, any other suitable clause, or any combination of suitable clauses, wherein the naloxone and the polymer/copolymer are present at a ratio of about 1:15 (naloxone:polymer/copolymer).
8. The nanoparticle composition of clause 2, any other suitable clause, or any combination of suitable clauses, wherein the naloxone and the polymer/copolymer are present at a ratio of about 1:20 (naloxone:polymer/copolymer).
9. The nanoparticle composition of clause 2, any other suitable clause, or any combination of suitable clauses, wherein the naloxone and the polymer/copolymer are present at a ratio of about 1:25 (naloxone:polymer/copolymer).
10. The nanoparticle composition of clause 2, any other suitable clause, or any combination of suitable clauses, wherein the naloxone and the polymer/copolymer are present at a ratio of about 0.1 mg:1 mg (naloxone:polymer/copolymer).
11. The nanoparticle composition of clause 2, any other suitable clause, or any combination of suitable clauses, wherein the naloxone and the polymer/copolymer are present at a ratio of about 1 mg:10 mg (naloxone:polymer/copolymer).
12. The nanoparticle composition of clause 2, any other suitable clause, or any combination of suitable clauses, wherein the naloxone and the polymer/copolymer are present at a ratio of about 0.5 mg:5 mg (naloxone:polymer/copolymer).
13. The nanoparticle composition of clause 1, any other suitable clause, or any combination of suitable clauses, wherein the ligand is gambogic acid.
14. The nanoparticle composition of clause 1, any other suitable clause, or any combination of suitable clauses, wherein the naloxone is encapsulated by the polymeric nanoparticle.
15. The nanoparticle composition of clause 1, any other suitable clause, or any combination of suitable clauses, wherein the nanoparticle composition has an average diameter from about 0.5 nm to about 1000 nm.
16. The nanoparticle composition of clause 1, any other suitable clause, or any combination of suitable clauses, wherein the nanoparticle composition has an average diameter from about 1 nm to about 500 nm.
17. The nanoparticle composition of clause 1, any other suitable clause, or any combination of suitable clauses, wherein the nanoparticle composition has an average diameter from about 10 nm to about 400 nm.
18. The nanoparticle composition of clause 1, any other suitable clause, or any combination of suitable clauses, wherein the nanoparticle composition has an average diameter from about 100 nm to about 400 nm.
19. The nanoparticle composition of clause 1, any other suitable clause, or any combination of suitable clauses, wherein the nanoparticle composition has an average diameter from about 100 nm to about 300 nm.
20. The nanoparticle composition of clause 1, any other suitable clause, or any combination of suitable clauses, wherein the nanoparticle composition has an average diameter from about 100 nm to about 200 nm.
21. The nanoparticle composition of clause 1, any other suitable clause, or any combination of suitable clauses, wherein the nanoparticle composition has an average diameter from about 100 nm to about 150 nm.
22. The nanoparticle composition of clause 1, any other suitable clause, or any combination of suitable clauses, wherein the nanoparticle composition has an average diameter from about 200 nm to about 400 nm.
23. The nanoparticle composition of clause 1, any other suitable clause, or any combination of suitable clauses, wherein the nanoparticle composition has an average diameter from about 300 nm to about 400 nm.
24. The nanoparticle composition of clause 1, any other suitable clause, or any combination of suitable clauses, wherein the nanoparticle composition has an average diameter from about 150 nm to about 300 nm.
25. The nanoparticle composition of clause 1, any other suitable clause, or any combination of suitable clauses, wherein the nanoparticle composition has an average diameter from about 150 nm to about 200 nm.
26. The nanoparticle composition of clause 1, any other suitable clause, or any combination of suitable clauses, wherein the nanoparticle composition has an average diameter from about 200 nm to about 300 nm.
27. The nanoparticle composition of clause 1, any other suitable clause, or any combination of suitable clauses, wherein the nanoparticle composition has an average diameter from about 250 nm to about 300 nm.
28. The nanoparticle composition of clause 1, any other suitable clause, or any combination of suitable clauses, wherein the nanoparticle composition is lyophilized.
29. A pharmaceutical composition comprising a nanoparticle composition, wherein the nanoparticle composition comprises i) a polymeric nanoparticle, ii) one or more ligands conjugated to the polymeric nanoparticle, and iii) naloxone.
30. The pharmaceutical composition of clause 29, any other suitable clause, or any combination of suitable clauses, wherein the pharmaceutical composition is an oral formulation.
31. The pharmaceutical composition of clause 30, any other suitable clause, or any combination of suitable clauses, wherein the oral formulation is selected from the group consisting of a tablet, a capsule, a suspension, an emulsion, a syrup, a colloidal dispersion, a dispersion, and an effervescent composition.
32. The pharmaceutical composition of clause 30, any other suitable clause, or any combination of suitable clauses, wherein the oral formulation is a suspension.
33. The pharmaceutical composition of clause 30, any other suitable clause, or any combination of suitable clauses, wherein the oral formulation is a reconstitutable suspension.
34. The pharmaceutical composition of clause 29, any other suitable clause, or any combination of suitable clauses, wherein the pharmaceutical composition is a parenteral formulation.
35. The pharmaceutical composition of clause 34, any other suitable clause, or any combination of suitable clauses, wherein the parenteral formulation is selected from the group consisting of intravenous, intraarterial, intraperitoneal, intrathecal, intradermal, epidural, intracerebroventricular, intraurethral, intrasternal, intracranial, intratumoral, intramuscular and subcutaneous.
36. The pharmaceutical composition of clause 29, any other suitable clause, or any combination of suitable clauses, wherein the pharmaceutical composition comprises one or more pharmaceutically acceptable carriers.
37. The pharmaceutical composition of clause 29, any other suitable clause, or any combination of suitable clauses, wherein the pharmaceutical composition further comprises a second therapeutic agent.
38. The pharmaceutical composition of clause 29, any other suitable clause, or any combination of suitable clauses, wherein the pharmaceutical composition is formulated as a single dose.
39. The pharmaceutical composition of clause 29, any other suitable clause, or any combination of suitable clauses, wherein the pharmaceutical composition is formulated as a single unit dose.
40. The pharmaceutical composition of clause 29, any other suitable clause, or any combination of suitable clauses, wherein the polymeric nanoparticle comprises a polymer/copolymer selected from the group consisting of polylactide, poly(lactide-co-glycolide), polycaprolactone, and any combination thereof.
41. The pharmaceutical composition of clause 40, any other suitable clause, or any combination of suitable clauses, wherein the naloxone and the polymer/copolymer are present at a ratio of about 1:4 (naloxone:polymer/copolymer).
42. The pharmaceutical composition of clause 40, any other suitable clause, or any combination of suitable clauses, wherein the naloxone and the polymer/copolymer are present at a ratio of about 1:5 (naloxone:polymer/copolymer).
43. The pharmaceutical composition of clause 40, any other suitable clause, or any combination of suitable clauses, wherein the naloxone and the polymer/copolymer are present at a ratio of about 1:10 (naloxone:polymer/copolymer).
44. The pharmaceutical composition of clause 40, any other suitable clause, or any combination of suitable clauses, wherein the naloxone and the polymer/copolymer are present at a ratio of about 1:12 (naloxone:polymer/copolymer).
45. The pharmaceutical composition of clause 40, any other suitable clause, or any combination of suitable clauses, wherein the naloxone and the polymer/copolymer are present at a ratio of about 1:15 (naloxone:polymer/copolymer).
46. The pharmaceutical composition of clause 40, any other suitable clause, or any combination of suitable clauses, wherein the naloxone and the polymer/copolymer are present at a ratio of about 1:20 (naloxone:polymer/copolymer).
47. The pharmaceutical composition of clause 40, any other suitable clause, or any combination of suitable clauses, wherein the naloxone and the polymer/copolymer are present at a ratio of about 1:25 (naloxone:polymer/copolymer).
48. The pharmaceutical composition of clause 29, any other suitable clause, or any combination of suitable clauses, wherein the ligand is gambogic acid.
49. The pharmaceutical composition of clause 29, any other suitable clause, or any combination of suitable clauses, wherein the naloxone is encapsulated by the polymeric nanoparticle.
50. The pharmaceutical composition of clause 29, any other suitable clause, or any combination of suitable clauses, wherein the nanoparticle composition has an average diameter from about 0.5 nm to about 1000 nm.
51. The pharmaceutical composition of clause 29, any other suitable clause, or any combination of suitable clauses, wherein the nanoparticle composition has an average diameter from about 1 nm to about 500 nm.
52. The pharmaceutical composition of clause 29, any other suitable clause, or any combination of suitable clauses, wherein the nanoparticle composition has an average diameter from about 10 nm to about 400 nm.
53. The pharmaceutical composition of clause 29, any other suitable clause, or any combination of suitable clauses, wherein the nanoparticle composition has an average diameter from about 100 nm to about 400 nm.
54. The pharmaceutical composition of clause 29, any other suitable clause, or any combination of suitable clauses, wherein the nanoparticle composition has an average diameter from about 100 nm to about 300 nm.
55. The pharmaceutical composition of clause 29, any other suitable clause, or any combination of suitable clauses, wherein the nanoparticle composition has an average diameter from about 100 nm to about 200 nm.
56. The pharmaceutical composition of clause 29, any other suitable clause, or any combination of suitable clauses, wherein the nanoparticle composition has an average diameter from about 100 nm to about 150 nm.
57. The pharmaceutical composition of clause 29, any other suitable clause, or any combination of suitable clauses, wherein the nanoparticle composition has an average diameter from about 200 nm to about 400 nm.
58. The pharmaceutical composition of clause 29, any other suitable clause, or any combination of suitable clauses, wherein the nanoparticle composition has an average diameter from about 300 nm to about 400 nm.
59. The pharmaceutical composition of clause 29, any other suitable clause, or any combination of suitable clauses, wherein the nanoparticle composition has an average diameter from about 150 nm to about 300 nm.
60. The pharmaceutical composition of clause 29, any other suitable clause, or any combination of suitable clauses, wherein the nanoparticle composition has an average diameter from about 150 nm to about 200 nm.
61. The pharmaceutical composition of clause 29, any other suitable clause, or any combination of suitable clauses, wherein the nanoparticle composition has an average diameter from about 200 nm to about 300 nm.
62. The pharmaceutical composition of clause 29, any other suitable clause, or any combination of suitable clauses, wherein the nanoparticle composition has an average diameter from about 250 nm to about 300 nm.
63. A method of treating opioid induced respiratory depression in a patient in need thereof, said method comprising the step of administering a therapeutically effective amount of a nanoparticle composition to the patient, wherein the nanoparticle composition comprises i) a polymeric nanoparticle, ii) one or more ligands conjugated to the polymeric nanoparticle, and iii) naloxone.
64. A method of treating opioid use disorder in a patient in need thereof, said method comprising the step of administering a therapeutically effective amount of a nanoparticle composition to the patient, wherein the nanoparticle composition comprises i) a polymeric nanoparticle, ii) one or more ligands conjugated to the polymeric nanoparticle, and iii) naloxone.
65. A method of treating alcoholism in a patient in need thereof, said method comprising the step of administering a therapeutically effective amount of a nanoparticle composition to the patient, wherein the nanoparticle composition comprises i) a polymeric nanoparticle, ii) one or more ligands conjugated to the polymeric nanoparticle, and iii) naloxone.
66. A method of treating opioid overdose in a patient in need thereof, said method comprising the step of administering a therapeutically effective amount of a nanoparticle composition to the patient, wherein the nanoparticle composition comprises i) a polymeric nanoparticle, ii) one or more ligands conjugated to the polymeric nanoparticle, and iii) naloxone.
67. A method of treating post-operative opioid depression in a patient in need thereof, said method comprising the step of administering a therapeutically effective amount of a nanoparticle composition to the patient, wherein the nanoparticle composition comprises i) a polymeric nanoparticle, ii) one or more ligands conjugated to the polymeric nanoparticle, and iii) naloxone.
68. A method of treating hypertension in a patient in need thereof, said method comprising the step of administering a therapeutically effective amount of a nanoparticle composition to the patient, wherein the nanoparticle composition comprises i) a polymeric nanoparticle, ii) one or more ligands conjugated to the polymeric nanoparticle, and iii) naloxone.
69. The method of clause 68, wherein the hypertension is associated with management of septic shock in the patient.
70. A method of treating pruritus in a patient in need thereof, said method comprising the step of administering a therapeutically effective amount of a nanoparticle composition to the patient, wherein the nanoparticle composition comprises i) a polymeric nanoparticle, ii) one or more ligands conjugated to the polymeric nanoparticle, and iii) naloxone.
71. The method of clause 70, wherein the pruritus is opioid-induced pruritus.
72. A method of preventing urinary retention in a patient, said method comprising the step of administering a therapeutically effective amount of a nanoparticle composition to the patient, wherein the nanoparticle composition comprises i) a polymeric nanoparticle, ii) one or more ligands conjugated to the polymeric nanoparticle, and iii) naloxone.
73. The method of clause 72, wherein the patient is a post-operative patient.
74. The method of clause 73, wherein the post-operative patient utilizes a patient controlled analgesia (PCA) device.
75. The method of any one of clauses 63 to 74, any other suitable clause, or any combination of suitable clauses, wherein the patient is an animal.
76. The method of clause 75, any other suitable clause, or any combination of suitable clauses, wherein the animal is a mammal.
77. The method of clause 75, any other suitable clause, or any combination of suitable clauses, wherein the animal is a human.
78. The method of any one of clauses 63 to 74, any other suitable clause, or any combination of suitable clauses, wherein the nanoparticle composition is administered to the patient at a dose of about 0.001 to about 1000 mg of the nanoparticle composition per kg of body weight.
79. The method of any one of clauses 63 to 74, any other suitable clause, or any combination of suitable clauses, wherein the nanoparticle composition is administered to the patient at a dose of about 0.001 to about 100 mg of the nanoparticle composition per kg of body weight.
80. The method of any one of clauses 63 to 74, any other suitable clause, or any combination of suitable clauses, wherein the nanoparticle composition is administered to the patient at a dose of about 0.001 to about 10 mg of the nanoparticle composition per kg of body weight.
81. The method of any one of clauses 63 to 74, any other suitable clause, or any combination of suitable clauses, wherein the nanoparticle composition is administered to the patient at a dose of about 1 to about 5 mg of the nanoparticle composition per kg of body weight.
82. The method of any one of clauses 63 to 74, any other suitable clause, or any combination of suitable clauses, wherein the nanoparticle composition is administered to the patient at a dose of about 1 mg of the nanoparticle composition per kg of body weight.
83. The method of any one of clauses 63 to 74, any other suitable clause, or any combination of suitable clauses, wherein the nanoparticle composition is administered to the patient at a dose of about 2 mg of the nanoparticle composition per kg of body weight.
84. The method of any one of clauses 63 to 74, any other suitable clause, or any combination of suitable clauses, wherein the nanoparticle composition is administered to the patient at a dose of about 3 mg of the nanoparticle composition per kg of body weight.
85. The method of any one of clauses 63 to 74, any other suitable clause, or any combination of suitable clauses, wherein the nanoparticle composition is administered to the patient at a dose of about 4 mg of the nanoparticle composition per kg of body weight.
86. The method of any one of clauses 63 to 74, any other suitable clause, or any combination of suitable clauses, wherein the nanoparticle composition is administered to the patient at a dose of about 5 mg of the nanoparticle composition per kg of body weight.
87. The method of any one of clauses 63 to 74, any other suitable clause, or any combination of suitable clauses, wherein the administration is an oral administration.
88. The method of clause 87, any other suitable clause, or any combination of suitable clauses, wherein the oral administration is selected from the group consisting of a tablet, a capsule, a suspension, an emulsion, a syrup, a colloidal dispersion, a dispersion, and an effervescent composition.
89. The method of clause 87, any other suitable clause, or any combination of suitable clauses, wherein the oral formulation is a suspension.
90. The method of clause 87, any other suitable clause, or any combination of suitable clauses, wherein the oral formulation is a reconstitutable suspension.
91. The method of any one of clauses 63 to 74, any other suitable clause, or any combination of suitable clauses, wherein the administration is a parenteral administration.
92. The method of clause 91, any other suitable clause, or any combination of suitable clauses, wherein the parenteral administration is selected from the group consisting of intravenous, intraarterial, intraperitoneal, intrathecal, intradermal, epidural, intracerebroventricular, intraurethral, intrasternal, intracranial, intratumoral, intramuscular and subcutaneous.
93. The method of any one of clauses 63 to 74, any other suitable clause, or any combination of suitable clauses, wherein the nanoparticle composition is administered as a single dose.
94. The method of any one of clauses 63 to 74, any other suitable clause, or any combination of suitable clauses, wherein the nanoparticle composition is administered as a single unit dose.
95. The method of any one of clauses 63 to 74, any other suitable clause, or any combination of suitable clauses, wherein the nanoparticle composition is administered to the patient once daily.
96. The method of any one of clauses 63 to 74, any other suitable clause, or any combination of suitable clauses, wherein the nanoparticle composition is administered to the patient twice daily.
97. The method of any one of clauses 63 to 74, any other suitable clause, or any combination of suitable clauses, wherein the nanoparticle composition is administered to the patient four times per week.
98. The method of any one of clauses 63 to 74, any other suitable clause, or any combination of suitable clauses, wherein the nanoparticle composition is administered to the patient three times per week.
99. The method of any one of clauses 63 to 74, any other suitable clause, or any combination of suitable clauses, wherein the nanoparticle composition is administered to the patient two times per week.
100. The method of any one of clauses 63 to 74, any other suitable clause, or any combination of suitable clauses, wherein the nanoparticle composition is administered to the patient one time per week.
101. The method of any one of clauses 63 to 74, any other suitable clause, or any combination of suitable clauses, wherein the nanoparticle composition is administered to the patient every 10 days.
102. The method of any one of clauses 63 to 74, any other suitable clause, or any combination of suitable clauses, wherein the nanoparticle composition is administered to the patient every 14 days.
103. The method of any one of clauses 63 to 74, any other suitable clause, or any combination of suitable clauses, wherein the nanoparticle composition is administered to the patient every 15 days.
104. The method of any one of clauses 63 to 74, any other suitable clause, or any combination of suitable clauses, wherein the nanoparticle composition is administered to the patient every 21 days.
105. The method of any one of clauses 63 to 74, any other suitable clause, or any combination of suitable clauses, wherein the nanoparticle composition is administered to the patient every 28 days.
106. The method of any one of clauses 63 to 74, any other suitable clause, or any combination of suitable clauses, wherein the nanoparticle composition is administered to the patient one time per month.
107. The method of any one of clauses 63 to 74, any other suitable clause, or any combination of suitable clauses, wherein the nanoparticle composition provides an onset of action to the patient in about 5 minutes.
108. The method of any one of clauses 63 to 74, any other suitable clause, or any combination of suitable clauses, wherein the nanoparticle composition provides an onset of action to the patient in about 10 minutes.
109. The method of any one of clauses 63 to 74, any other suitable clause, or any combination of suitable clauses, wherein the nanoparticle composition provides an onset of action to the patient in about 15 minutes.
110. The method of any one of clauses 63 to 74, any other suitable clause, or any combination of suitable clauses, wherein the nanoparticle composition provides an onset of action to the patient in about 20 minutes.
111. The method of any one of clauses 63 to 74, any other suitable clause, or any combination of suitable clauses, wherein the nanoparticle composition provides an onset of action to the patient in about 25 minutes.
112. The method of any one of clauses 63 to 74, any other suitable clause, or any combination of suitable clauses, wherein the nanoparticle composition provides an onset of action to the patient in about 30 minutes.
113. The method of any one of clauses 63 to 74, any other suitable clause, or any combination of suitable clauses, wherein the nanoparticle composition provides an onset of action to the patient in about 35 minutes.
114. The method of any one of clauses 63 to 74, any other suitable clause, or any combination of suitable clauses, wherein the nanoparticle composition provides an onset of action to the patient in about 40 minutes.
115. The method of any one of clauses 63 to 74, any other suitable clause, or any combination of suitable clauses, wherein the nanoparticle composition provides an onset of action to the patient in about 45 minutes.
116. The method of any one of clauses 63 to 74, any other suitable clause, or any combination of suitable clauses, wherein the nanoparticle composition provides an onset of action to the patient in about 60 minutes.
117. The method of any one of clauses 63 to 74, any other suitable clause, or any combination of suitable clauses, wherein the method further comprises administration of a second therapeutic agent to the patient.
118. The method of any one of clauses 63 to 74, any other suitable clause, or any combination of suitable clauses, wherein the polymeric nanoparticle comprises a polymer/copolymer selected from the group consisting of polylactide, poly(lactide-co-glycolide), polycaprolactone, and any combination thereof.
119. The method of any one of clauses 63 to 74, any other suitable clause, or any combination of suitable clauses, wherein the ligand is gambogic acid.
120. The method of any one of clauses 63 to 74, any other suitable clause, or any combination of suitable clauses, wherein the naloxone is encapsulated by the polymeric nanoparticle.
121. The method of any one of clauses 63 to 74, any other suitable clause, or any combination of suitable clauses, wherein the nanoparticle composition has an average diameter from about 0.5 nm to about 1000 nm.
122. The method of any one of clauses 63 to 74, any other suitable clause, or any combination of suitable clauses, wherein the nanoparticle composition has an average diameter from about 1 nm to about 500 nm.
123. The method of any one of clauses 63 to 74, any other suitable clause, or any combination of suitable clauses, wherein the nanoparticle composition has an average diameter from about 10 nm to about 400 nm.
124. The method of any one of clauses 63 to 74, any other suitable clause, or any combination of suitable clauses, wherein the nanoparticle composition has an average diameter from about 100 nm to about 400 nm.
125. The method of any one of clauses 63 to 74, any other suitable clause, or any combination of suitable clauses, wherein the nanoparticle composition has an average diameter from about 100 nm to about 300 nm.
126. The method of any one of clauses 63 to 74, any other suitable clause, or any combination of suitable clauses, wherein the nanoparticle composition has an average diameter from about 100 nm to about 200 nm.
127. The method of any one of clauses 63 to 74, any other suitable clause, or any combination of suitable clauses, wherein the nanoparticle composition has an average diameter from about 100 nm to about 150 nm.
128. The method of any one of clauses 63 to 74, any other suitable clause, or any combination of suitable clauses, wherein the nanoparticle composition has an average diameter from about 200 nm to about 400 nm.
129. The method of any one of clauses 63 to 74, any other suitable clause, or any combination of suitable clauses, wherein the nanoparticle composition has an average diameter from about 300 nm to about 400 nm.
130. The method of any one of clauses 63 to 74, any other suitable clause, or any combination of suitable clauses, wherein the nanoparticle composition has an average diameter from about 150 nm to about 300 nm.
131. The method of any one of clauses 63 to 74, any other suitable clause, or any combination of suitable clauses, wherein the nanoparticle composition has an average diameter from about 150 nm to about 200 nm.
132. The method of any one of clauses 63 to 74, any other suitable clause, or any combination of suitable clauses, wherein the nanoparticle composition has an average diameter from about 200 nm to about 300 nm.
133. The method of any one of clauses 63 to 74, any other suitable clause, or any combination of suitable clauses, wherein the nanoparticle composition has an average diameter from about 250 nm to about 300 nm.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

### Example 1

### Nanoparticle Composition Polymer Synthesis and Characterization

In the instant example, exemplary polymers for the nanoparticle compositions were prepared. Polymer was synthesized through a two-step condensation method. Briefly, step 1 involved preparation of pre-polymers through solution, polycondensation of lactide dimer (12 g; 0.0833 mol) and polyethylene glycol (PEG, MW 400, 3.52 g; 1.744 mL; 0.0057 mol) in presence of stannous octoate catalyst (12 μL; 0.1% w/w) and toluene (50 mL). The reactants were refluxed over 24 h at 115° C. under stirring in presence of nitrogen. The reaction mixture was cooled to room temperature and the solvent was evaporated under reduced pressure.

The resultant residue was then dissolved in 12 mL of CH₂Cl₂ and precipitated in 400 mL of cold diethyl ether under vigorous stirring. The pre-polymer product was obtained by centrifugation (5000 rpm and 4° C. for 10 min) tracked by decantation followed by vacuum drying to a constant weight (10 g, 70% yield).

Step 2 involved chain extension of pre-polymers (10 g; 1.63 mmol) via reactive linker cyclohexane-tetracarboxylic dianhydride (HCDA, 365.12 mg; 1.63 mmol) in presence of trimethylamine (TEA, 454 μL; 3.26 mmol) at 1:1:2 molar ratio. The reactants were refluxed with toluene (50 mL) over 24 h at 115° C. under stirring in presence of nitrogen, stannous octoate (15 μL; 0.15% w/w) and subsequently the reaction mixture was cooled and solvent was evaporated under reduced pressure. The resultant residue was dissolved in 10 mL CH₂Cl₂ and precipitated in 400 mL cold diethyl ether under vigorous stirring.

The polymer was collected by centrifugation (5000 rpm and 4° C. for 10 min) and decantation followed by vacuum drying to a constant dry weight (7 g, 70% yield). The pre-polymer and polymer are characterized by ¹H NMR, ¹³C NMR and GPC. The polymer was made reproducibly in large quantities of approximately 10-15 g.

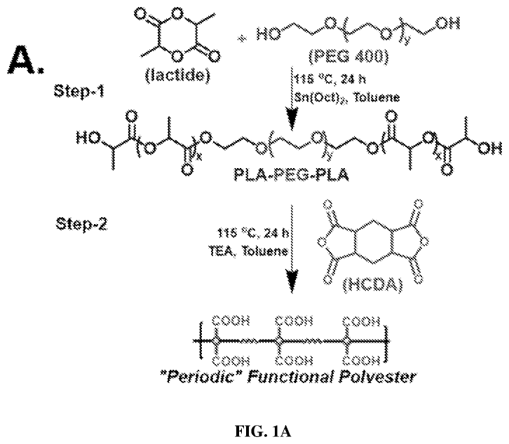

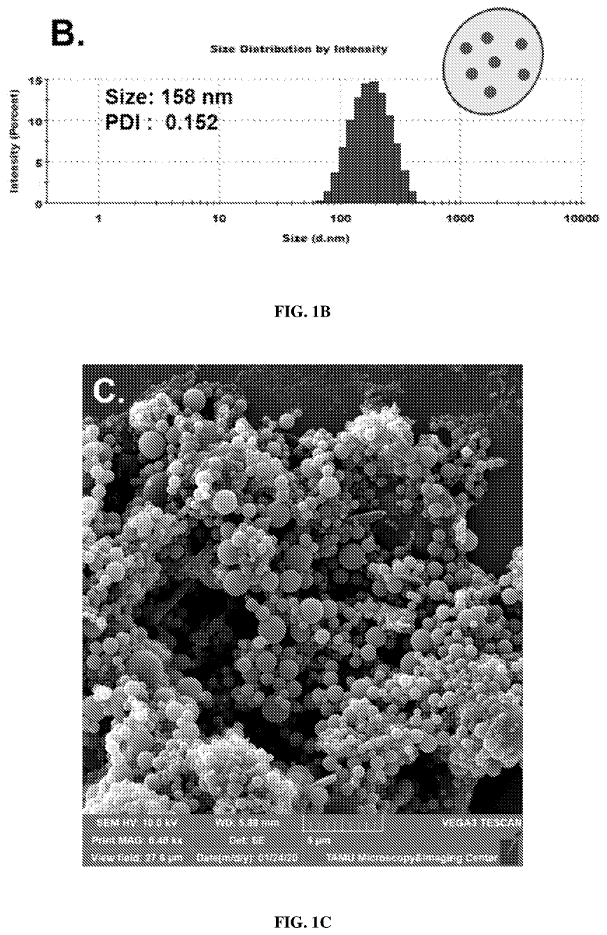

The ¹H NMR of pre-polymer (PLA-PEG400) revealed the PLA methyl and methylene protons peaks at 1.55-1.58 ppm and 5.11-5.15 ppm respectively, and PEG methylene proton peak at 3.60-3.62 ppm. While the peaks at 3.65 and 4.20-4.30 ppm are due to PEG methylene linked to hydroxyl ends and to the PLA unit junctions. A combination of ¹H NMR and ¹³C NMR were used to characterize the final polymer as the linker HCDA peaks in 1H NMR are buried under PLA peaks. The peaks at 21, 40 and 175 ppm in ¹³C NMR are characteristic of the linker HCDA. The molecular weight of the polymer was analyzed by gel permeation chromatography (GPC) and found to be Mn=12,000 and Mw=30,000. The schematic of polymer synthesis is shown in FIG. 1A.

### Example 2

### Naloxone Nanoparticle Composition Preparation and Characterization

### Preparation and Characterization

Nanoparticle compositions comprising naloxone (also referred to herein as “NP Naloxone”) were prepared in the instant example. Briefly, the organic phase included polymer (50 mg in 2 mL of ethyl acetate) and naloxone (5 mg, dissolved in 0.5 mL of ethyl acetate+50 uL of DMSO; Sigma-Aldrich, St. Louis, Mo.) stirred separately for 30 minutes and later mixed and stirred for next 30 minutes at 1000 rpm. The aqueous phase included vitamin ETPGS 50 mg dissolved in 5 mL of water. The organic phase was emulsified into aqueous phase under stirring at 1500 rpm for 1 minute followed by sonication at 30% amplitude for 45 seconds. The organic solvent was evaporated under continuous stirring at room temperature and the particles were collected as pellet by centrifugation at 15000 g, 30 minutes, 4° C. The pellet was re-suspended in 5% trehalose solution in water and freeze dried. The freeze drying was carried out using bench top freeze drier (Labconco® FreeZone® Triad® −85° C. Benchtop Freeze Dryers) at −55° C. for 54 h, followed heating at 20° C. for 20 h under vacuum (0.008 mbar). The freeze product was crimp sealed and stored at 4° C. until further use.

The particles were characterized for size, morphology and entrapment efficiency. The entrapment efficiency was determined by HPLC isocratic method, A-acetonitrile and B-12.5 mM phosphate buffer (A-70%; B-30%) with flow rate of 0.8 mL/minute, injection volume 5 μL, and detection was performed with UV detector at 220 nm. The retention time of the naloxone was 2.8 minutes. The lowest limit of naloxone detection was set to be 5 μg, while this suits the current study design which is deemed to study only the entrapment efficiency; however, conducting a PK study would need LOD to be established. Illustration of the nanoparticle composition properties are shown in FIGS. 1A-C.

The nanoparticle compositions, with and without naloxone, are in the size range of 150 nm and scanning electron micrographs reveals that the particles are in spherical shape. The nanoparticle composition preparation method provided 0.1 mg of naloxone per 1 mg of polymer. The freeze drying procedures did not alter the nanoparticle composition characteristics, compared to fresh preparations. FIGS. 1B-1C show the distribution of the nanoparticle compositions by intensity and particle shapes.

### Animal Testing

All procedures were conducted in accordance with the National Institutes of Health Guide for the Care and Use of Laboratory Animals, and were approved by the Institutional Animal Care and Use Committee. Male C57BL/6N mice were purchased from Envigo Lab (Houston, Tex., USA) and housed four per cage with food and water ad libitum in a temperature-controlled (21+/−2° C., humidity 45%) vivarium with a 12-hour light/12-hour dark cycle (light on at 7:30 AM). All mice were housed as cage-mates from the day of weaning (postnatal day (PND) 21) in Envigo facilities, shipped as cage-mates and remained cage-mates four mice per cage after arriving at our facility and for the entire experiment.

### Example 3

### Duration of Action of Nanoparticle Compositions In Vivo

### Antagonizing Morphine-Induced Locomotion

The instant procedure was conducted in a set of eight identical Photobeam Activity System (San Diego Instruments, San Diego, Calif.). A multiplexor-analyzer was interfaced with a PC and simultaneously tracked the interruption of beams from the optical beam activity monitors. The animal position was updated every 10 ms. The integration of the data (PAS Reporter) about the location of the animal was used to determine the distance traveled (in cm).

Mice were habituated to the room for at least 30 minutes prior to testing and then placed separately (one mouse per apparatus) in the Photobeam Activity System. Mice were physically and visually separated during the locomotion test. Baseline locomotion activity was recorded for 30 minutes. Then, mice were administered orally (via gavage, 10 mg/ml) with control nanoparticle compositions (i.e., no active ingredient; “NP control”) (n=8), 1 mg/kg NP Naloxone (n=8), or 5 mg/kg NP Naloxone (n=8).

Immediately after (T0), mice were injected with 10 mg/kg morphine (s.c., 10 mg/ml) and recorded for 3 hours. At 10 hours (T10), 24 hours (T24), 34 hours (T34), 48 hours (T48), 72 hours (T72), and 96 hours (T96) following the nanoparticle composition administration, each mouse was re-injected with saline or morphine, place in the activity box, and recorded for 3 hours. Mice were returned to their home cages between recordings. The apparatus was thoroughly cleaned with ethanol and water between recordings.

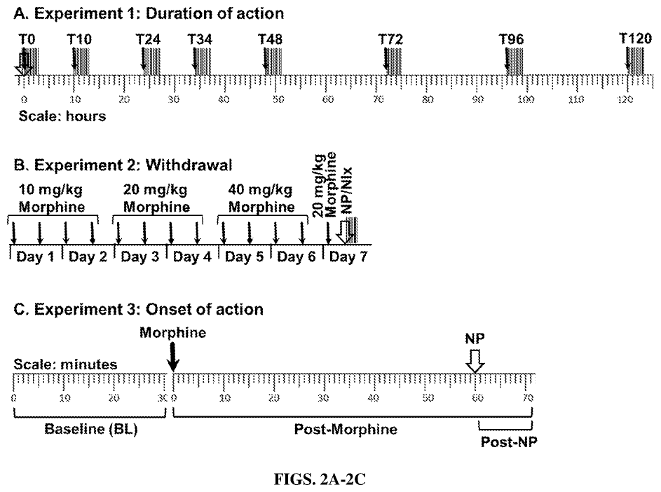

As shown in FIG. 2A, baseline (BL) locomotor activity of each mouse was recorded for 30 minutes. Then, mice were administered orally (via gavage, 10 ml/kg) with NP control (n=8), 1 mg/kg NP Naloxone (n=8), or 5 mg/kg NP Naloxone (n=8). Immediately after (T0), they were administered 10 mg/kg morphine (10 ml/kg, s.c.) and recorded for another 3 hours. Subsequently, mice were re-injected with 10 mg/kg morphine 10 hours (T10), 24 hours (T24), 34 hours (T34), 48 hours (T48), 72 hours (T72), and 96 hours (T96) following the NP administration. Each time the mice were recorded for 3 hours.

One-way ANOVA for the total distance traveled during the baseline period revealed significant main effects of group (F(2, 21)=13.2, p<0.0001). Similarly, two-way repeated ANOVA for the 5-minute intervals during the baseline revealed significant main effects of time (F(5, 105)=51.6, p<0.0001), and group (F(2, 21)=13.2, p<0.0001), and a significant interaction between time and group (F(10, 105)=14.4, p<0.0001). Bonferroni post hoc contrast for the total distance traveled revealed that the NP control group locomote significantly less than both the 1 mg/kg NP Naloxone (p<0.0001) and 5 mg/kg NP Naloxone (p<0.001) groups. Although the magnitude of these differences were minute, nonetheless, for the following analyses, the total distance traveled during the baseline period, or the distance traveled during the last 5-minute interval of baseline (referred to as t25-t30), were used as covariates.

Two-way repeated ANOVA (with BL as covariate) revealed a significant main effect of group (F(2, 20)=5.6, p<0.05), and a significant interaction between period and group (F(12, 120)=4.3, p<0.0001). Bonferroni post hoc contrast revealed that the NP control group locomote significantly more than the 1 mg/kg NP Naloxone group at T0 (p<0.0001) and T24 (p<0.05). Additionally, the NP control group locomoted significantly more than the 5 mg/kg NP Naloxone at T0 (p<0.01), T24 (p<0.05), and T34 (p<0.0001).

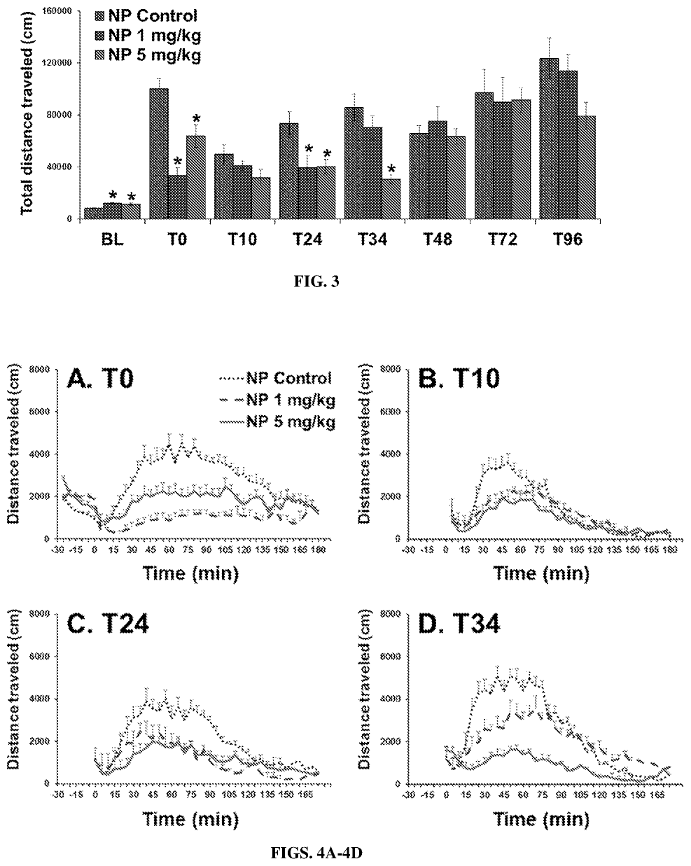

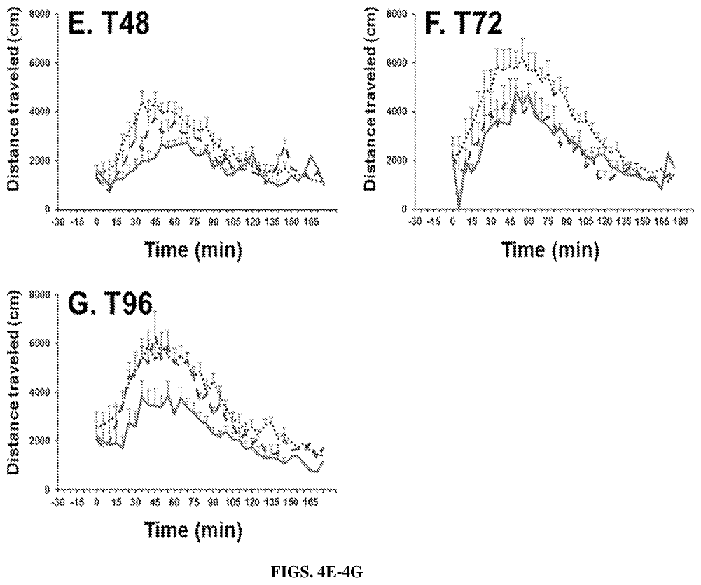

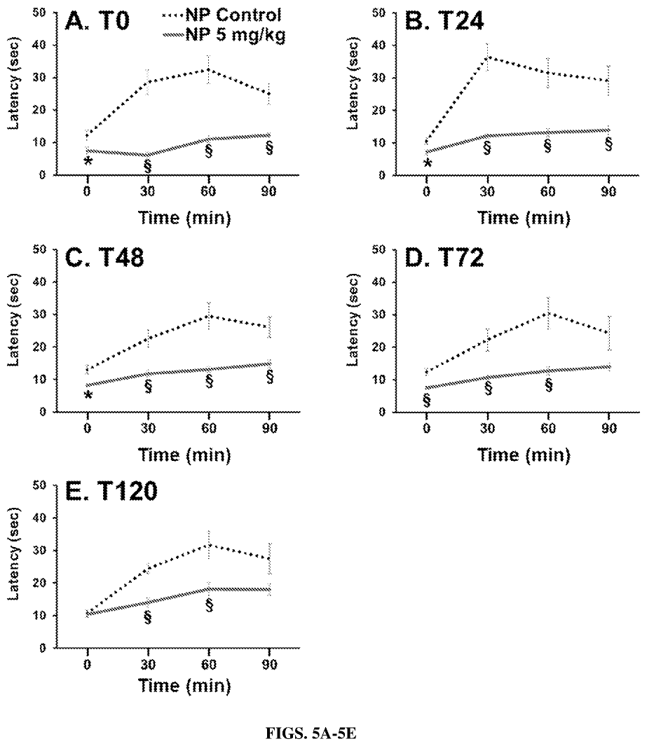

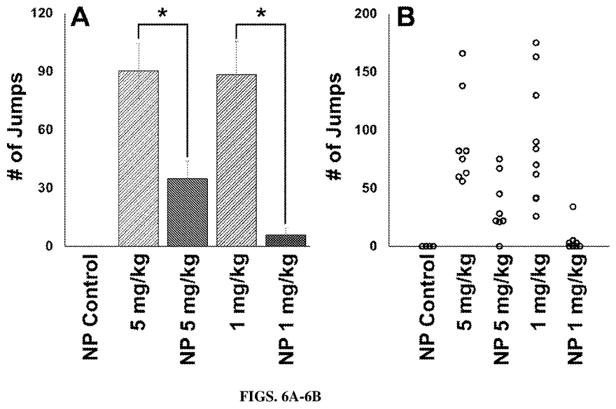

Separate analyses were also performed for the 3 hour periods following each of the morphine administrations (see FIG. 3). For T0 (FIG. 3, FIG. 4A), one-way ANOVA for the total distance traveled during the 3 hours post-morphine administration (with baseline as covariate) revealed a significant main effect of group (F(2, 20)=13.0, p<0.0001). Two-way repeated ANOVA for the 5-minute intervals (with t25-t30 as covariate) revealed a significant main effect of group (F(2, 20)=11.0, p<0.001), and a significant interactions between time and group (F(70, 700)=4.2, p<0.0001). Bonferroni post hoc contrast for the total distance traveled revealed that the NP control group locomote significantly more than both the 1 mg/kg NP Naloxone (p<0.0001) and the 5 mg/kg NP Naloxone (p<0.01) groups.

For T10 (FIG. 3, FIG. 4B), one-way ANOVA for the total distance traveled during the 3 hours post-morphine administration (with BL as covariate) revealed a significant main effect of group (F(2, 20)=4.7, p<0.05). Two-way repeated ANOVA for the 5-minute intervals (with t25-t30 as covariate) revealed a significant interaction between time and group (F(70, 700)=3.1, p<0.0001). During six of the 5-minute intervals ranging from 55 to 90 minutes post-morphine administration, Bonferroni post hoc contrast revealed that the NP control group locomote significantly more than both the 1 mg/kg NP Naloxone (p<0.05) and 5 mg/kg NP Naloxone (p<0.05) groups.

For T24 (FIG. 3, FIG. 4C), one-way ANOVA for the total distance traveled during the 3 hours post-morphine administration (with BL as covariate) revealed a significant main effect of group (F(2, 20)=6.2, p<0.01). Two-way repeated ANOVA for the 5-minute intervals (with t25-t30 as covariate) revealed significant main effects of time (F(35, 700)=2.3, p<0.0001) and group (F(2, 20)=4.5, p<0.05), and a significant interaction between time and group (F(70, 700)=2.0, p<0.0001). Bonferroni post hoc contrast for the total distance traveled revealed that the NP control group locomoted significantly more than the 1 mg/kg NP Naloxone (p<0.05) and 5 mg/kg NP Naloxone (p<0.05) groups.

For T34 (FIG. 3, FIG. 4D), one-way ANOVA for the total distance traveled during the 3 hours post-morphine administration (with BL as covariate) revealed a significant main effect of group (F(2, 20)=10.9, p<0.001). Two-way repeated ANOVA for the 5-minute intervals (with t25-t30 as covariate) revealed significant main effects of time (F(35, 700)=1.5, p<0.05) and group (F(2, 20)=13.1, p<0.0001), and a significant interaction between time and group (F(70, 700)=7.7, p<0.0001). Bonferroni post hoc contrast for the total distance traveled revealed that the NP control group locomoted significantly more than 5 mg/kg NP Naloxone (p<0.0001). Additionally, during eleven of the 5-minute intervals ranging from 55 to 115 minutes post-morphine administration, Bonferroni post hoc contrast revealed that NP control group locomoted significantly more than the 1 mg/kg NP Naloxone group (p<0.05).

For T48 (FIG. 3, FIG. 4E), one-way ANOVA for the total distance traveled during the 3 hours post-morphine administration (with BL as covariate) revealed no significant main effect of group (F(2, 20)=0.6, p>0.05). However, two-way repeated ANOVA for the 5-minute intervals (with t25-t30 as covariate) revealed a significant main effect of time (F(35, 700)=1.5, p<0.05) and a significant interaction between time and group (F(70, 700)=2.7, p<0.0001). During three of the 5-minute intervals ranging from 70 to 85 minutes post-morphine administration, Bonferroni post hoc contrast revealed that NP control group locomoted significantly more than the 5 mg/kg NP Naloxone group (p<0.05). Additionally, during the 5-minute internal of 70 to 75 minutes post-morphine administration, NP control group locomoted significantly more than the 1 mg/kg NP Naloxone (p<0.05).

For T72 (FIG. 3, FIG. 4F), one-way ANOVA for the total distance traveled during the 3 hours post-morphine administration (with BL as covariate) revealed no significant main effect of group (F(2, 20)=1.5, p>0.05). However, two-way repeated ANOVA for the 5-minute intervals (with t25-t30 as covariate) revealed a significant interaction between time and group (F(70, 700)=1.5, p<0.01). No significant post hoc contrasts were found.

For T96 (FIG. 3, FIG. 4G), one-way ANOVA for the total distance traveled during the 3 hours post-morphine administration (with BL as covariate) revealed a significant main effect of group (F(2, 20)=5.7, p<0.05). Two-way repeated ANOVA for the 5-minute intervals (with t25-t30 as covariate) revealed significant main effects of time (F(35, 700)=1.8, p<0.01) and group (F(2, 20)=4.0, p<0.05), and a significant interaction between time and group (F(70, 700)=1.4, p<0.05). Bonferroni post hoc contrast for the total distance traveled revealed a trend for the NP control group to locomote more than 5 mg/kg NP Naloxone (p=0.057). Additionally, during three of the 5-minute intervals ranging from 55 to 70 minutes post-morphine administration, Bonferroni post hoc contrast revealed that NP control group locomoted significantly more than the 5 mg/kg NP Naloxone group (p<0.05).

### Antagonizing Morphine-Induced Antinociception

Mice were habituated to the room for at least 30 minutes prior to testing. Mice were placed in a Plexiglas cylinder atop a hot plate apparatus. The surface temperature was maintained at 55±1° C. Licking of one of the hindpaws or jumping was taken as the animal's response to the painful stimulus, and the latency to perform one of these responses was recorded in seconds. Maximum cut-off time was set at 60 seconds to prevent tissue damage.

Baseline latency to respond (in seconds) was recorded. Then, mice were administered orally (via gavage, 10 ml/kg) with NP control (n=8) or 5 mg/kg NP Naloxone (n=8). Immediately afterwards (T0), mice were administered 10 mg/kg morphine (10 ml/kg, s.c.). At time points of 30, 60 and 90 minutes after morphine administration, mice were re-placed atop a hot plate apparatus and recorded for their latency to respond (in seconds). Subsequently, at 24 hours (T24), 48 hours (T48), 72 hours (T72), and 120 hours (T120) following the nanoparticle composition administration, baselines for latency to respond (in seconds) were re-recorded. Then, each time, mice were re-injected with 10 mg/kg morphine, and recorded for their latency to respond (in seconds) at 30, 60 and 90 minutes after morphine administration.

As shown in FIG. 2A, Mice were administered orally (via gavage, 10 ml/kg) with NP control (n=8) or 5 mg/kg NP Naloxone (n=8). The ability of NP Naloxone to antagonize the antinociceptive effects of 10 mg/kg morphine was examined immediately after (T0), 24 hours (T24), 48 hours (T48), 72 hours (T72), and 120 hours (T120) following the NP administration.

Two-way repeated ANOVAs revealed significant main effects of time and group and significant interactions between time and group at T0 (time: F(3, 42)=17.20, p<0.0001; group: F(1, 14)=37.87, p<0.0001; time×group: F(3, 42)=11.72, p<0.0001; FIG. 5A), T24 (time: F(3, 42)=17.90, p<0.0001; group: F(1, 14)=42.94, p<0.0001; time×group: F(3, 42)=6.95, p<0.001; FIG. 5B), T48 (time: F(3, 42)=21.85, p<0.0001; group: F(1, 14)=22.95, p<0.0001; time×group: F(3, 42)=5.20, p<0.01; FIG. 5C), T72 (time: F(3, 42)=15.00, p<0.0001; group: F(1, 14)=13.24, p<0.01; time×group: F(3, 42)=4.17, p<0.05; FIG. 5D), and T120 (time: F(3, 42)=20.24, p<0.0001; group: F(1, 14)=13.26, p<0.01; time×group: F(3, 42)=4.02, p<0.05; FIG. 5E). Due to development of antinociceptive tolerance, a reduction in morphine effect was observed over time. Nonetheless, Bonferroni post hoc contrast revealed that, compared to NP Controls, 5 mg/kg NP Naloxone significantly antagonized the antinociceptive effect of morphine up to 120 hours post NP administration (FIGS. 5A-5E).

### Example 4

### Withdrawal Participation of Nanoparticle Compositions In Vivo

For Experiment 2 (see FIG. 2B), mice were subcutaneously injected twice daily (at approximately 9:00 a.m. and 5:00 p.m.) for six consecutive days with either saline or increasing doses of morphine. Specifically, on days 1 and 2, the mice were injected with saline or 10 mg/kg morphine. On days 3 and 4, mice were injected with saline or 20 mg/kg morphine. On days 5 and 6, mice were injected with saline or 40 mg/kg morphine. A volume of 10 ml/kg was used for the saline and morphine injections.

A final dose was administered on day 7 in which mice were injected with saline or 20 mg/kg morphine. Two hours later, mice were administered with saline (i.p., n=4), 1 mg/kg naloxone (i.p., n=10), 5 mg/kg naloxone (i.p., n=8), NP controls (gavage, n=4), 1 mg/kg NP Naloxone (gavage, n=8), or 5 mg/kg NP Naloxone (gavage, n=8). Immediately thereafter, mice were individually placed in Plexiglas cylinders (37 cm tall×14.5 cm in diameter) and were videotaped for 30 minutes. The videotapes were scored for number of jumps. A jump was observed to be all four legs simultaneously lifting off the floor (i.e., the mouse has no contact with the floor).

As shown in FIG. 2B, morphine-dependent mice were administered with saline (i.p., n=4), 1 mg/kg naloxone (i.p., n=10), 5 mg/kg naloxone (i.p., n=8), NP controls (gavage, n=4), 1 mg/kg NP Naloxone (gavage, n=8), 5 mg/kg NP Naloxone (gavage, n=8). They were immediately recorded for jumping behaviors for 30 minutes.

Two-way ANOVA revealed significant main effects of drug (F(1, 38)=17.2, p<0.0001) and dose (F(2, 38)=9.3, p<0.001), and a significant interaction between drug and dose (F(2, 38)=4.2, p<0.05). Bonferroni post hoc contrast revealed that both the 5 mg/kg and 1 mg/kg naloxone groups jumped significantly more than the control group (p<0.001, FIGS. 6A-B). However, 5 mg/kg NP Naloxone and 1 mg/kg NP Naloxone groups did not jump significantly more than NP control group. Moreover, 5 mg/kg NP Naloxone group jumped significantly less than the 5 mg/kg naloxone group (p<0.05, FIG. 6A-B). Similarly, the 1 mg/kg NP Naloxone group jumped significantly less than the 1 mg/kg group (p<0.0001, FIG. 6A-B).

### Example 5

### Onset of Action of Nanoparticle Compositions In Vivo

The experiment was conducted in a set of eight identical Photobeam Activity Systems (San Diego Instruments, San Diego, Calif.), as detailed for experiment 1. Mice were habituated to the room for at least 30 minutes prior to testing and then placed separately (one mouse per apparatus) in the Photobeam Activity System. Mice were physically and visually separated during the locomotion test. Baseline locomotion activity was recorded for 30 minutes. Then, mice were administered with 10 mg/kg morphine (s.c., 10 mg/ml) and recorded for 60 minutes. Mice were then administered orally (via gavage, 10 mg/ml) with NP control (n=8) or 1 mg/kg NP Naloxone (n=8) and recorded for 10 minutes. The apparatus was thoroughly cleaned with ethanol and water between recordings.

As shown in FIG. 2C, baseline (BL) locomotor activity of each mouse was recorded for 30 minutes. Then, mice were administered 10 mg/kg morphine (10 ml/kg, s.c.) and recorded for another 60 minutes. Mice were then administered orally (via gavage, 10 ml/kg) with NP control (n=8) or 1 mg/kg NP Naloxone (n=8) and recorded for another 10 minutes.

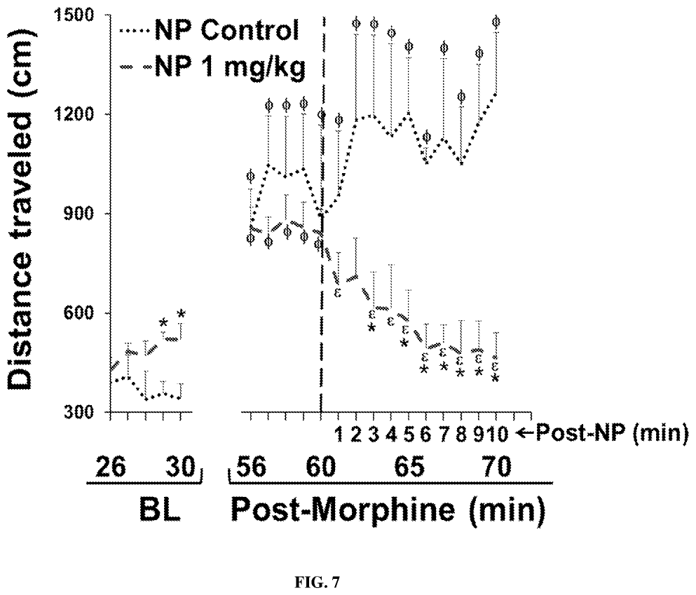

To reveal the onset of action of the 1 mg/kg NP Naloxone on locomotion, comparisons were made between the 1-minute intervals of the last 5 minutes of baseline (i.e. minutes 26-30 of baseline just prior to morphine administration), the last 5 minutes on morphine prior to NP administration (i.e. 56-60 minutes post-morphine administration), and the 10 minutes post-NP administration (FIG. 7).

Two way repeated ANOVA revealed significant main effects of time (F(19, 190)=12.4, p<0.0001) and group (F(1, 10)=9.3, p<0.05), and a significant interaction between time and group (F(19, 190)=7.8, p<0.0001). As expected, Bonferroni post hoc contrast revealed that morphine administration increased locomotor activity (minute 30 of BL vs. minutes 56-60 post-morphine, p<0.05). Administration of NP control did not significantly alter locomotor activity (minutes 60 post-morphine vs. 1-10 minutes post-NP administration, p>0.05, n.s.). In contrast, administration of 1 mg/kg NP Naloxone significantly reduced locomotor activity. This reduction could be already observed within 1 minute of administration (minute 60 post-morphine vs. 1 minute post-NP administration, p>0.01), and was fully established (back to baseline activity levels) within 5 minutes of administration.

1. A nanoparticle composition comprising i) a polymeric nanoparticle, ii) one or more ligands conjugated to the polymeric nanoparticle, and iii) naloxone.

2. The nanoparticle composition of claim 1, wherein the polymeric nanoparticle comprises a polymer/copolymer selected from the group consisting of polylactide, poly(lactide-co-glycolide), polycaprolactone, and any combination thereof.

3. The nanoparticle composition of claim 2, wherein the naloxone and the polymer/copolymer are present at a ratio of about 1:4 (naloxone:polymer/copolymer).

4. The nanoparticle composition of claim 2, wherein the naloxone and the polymer/copolymer are present at a ratio of about 1:5 (naloxone:polymer/copolymer).

5. The nanoparticle composition of claim 2, wherein the naloxone and the polymer/copolymer are present at a ratio of about 1:10 (naloxone:polymer/copolymer).

6. The nanoparticle composition of claim 1, wherein the ligand is gambogic acid.

7. (canceled)

8. (canceled)

9. The nanoparticle composition of claim 1, wherein the nanoparticle composition is lyophilized.

10. A pharmaceutical composition comprising a nanoparticle composition, wherein the nanoparticle composition comprises i) a polymeric nanoparticle, ii) one or more ligands conjugated to the polymeric nanoparticle, and iii) naloxone.

11. The pharmaceutical composition of claim 10, wherein the pharmaceutical composition is an oral formulation.

12. The pharmaceutical composition of claim 11, wherein the oral formulation is selected from the group consisting of a tablet, a capsule, a suspension, an emulsion, a syrup, a colloidal dispersion, a dispersion, and an effervescent composition.

13. The pharmaceutical composition of claim 11, wherein the oral formulation is a suspension.

14. The pharmaceutical composition of claim 11, wherein the oral formulation is a reconstitutable suspension.

15. (canceled)

16. (canceled)

17. The pharmaceutical composition of claim 10, wherein the polymeric nanoparticle comprises a polymer/copolymer selected from the group consisting of polylactide, poly(lactide-co-glycolide), polycaprolactone, and any combination thereof.

18. The pharmaceutical composition of claim 10, wherein the ligand is gambogic acid.

19. A method of treating opioid induced respiratory depression in a patient in need thereof, said method comprising the step of administering a therapeutically effective amount of a nanoparticle composition to the patient, wherein the nanoparticle composition comprises i) a polymeric nanoparticle, ii) one or more ligands conjugated to the polymeric nanoparticle, and iii) naloxone.

20. (canceled)

21. (canceled)

22. (canceled)

23. The method of claim 19, wherein the nanoparticle composition is administered to the patient at a dose of about 1 to about 5 mg of the nanoparticle composition per kg of body weight.

24. The method of claim 19, wherein the administration is an oral administration.

25. The method of claim 24, wherein the oral administration is selected from the group consisting of a tablet, a capsule, a suspension, an emulsion, a syrup, a colloidal dispersion, a dispersion, and an effervescent composition.

26. The method of claim 24, wherein the oral formulation is a suspension.

27. The method of claim 24, wherein the oral formulation is a reconstitutable suspension.

28.-36. (canceled)
